(19) United States
(12) Patent Application Publication
(10) Pub. No.: US 2007/0020723 A1
(43) Pub. Date: Jan. 25, 2007

(54) Ceramide kinase-like proteins

(76) Inventor: Frederic Bornancin, Wien (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/558,689

(22) PCT Filed: Apr. 7, 2004
(86) PCT No.: PCT/EP04/03740

§ 371 Date: Nov. 29, 2005

Related U.S. Application Data
(60) Provisional application No. 60/474,239, filed on May 29, 2003.

(51) Int. Cl. A61K 38/54; A61K 38/00; C07H 21/04; C12P 21/06; C12N 9/12

(52) U.S. Cl. 435/69.1; 435/194; 435/320.1; 435/325; 514/12; 536/23.2

(57) ABSTRACT

A ceramide kinase like protein encoded by a polynucleotide of SEQ ID NO: 1 to SEQ ID NO: 9 and its use.

The present invention relates to organic compounds, such as a protein, e.g. a ceramide kinase like protein.

Sphingolipids have been considered as one of the major components of the cell membrane. Recent evidence has shown that, beyond their structural role, they can act as bioactive lipids and impact on signal transduction, in a way that is reminiscent of what is occurring with glycerophospholipids.

Physiological activity of sphingolipid metabolites include e.g. induction of apoptosis and stimulation of cell proliferation and it has been suggested that enzymes which metabolise sphingolipids are expected to participate in the induction of various diseases.

For example

ceramide which controls cell mechanisms has been suggested to be a regulator in the enzymatic reaction indicated above, see e.g. Hannun, Y. A. et al, TIBS 20, 73-77 (1995);
Spiegel, S. et al, Curr. Opin. Cell. Biol. 8, 159-167 (1996) and Mathias S. et al, Science 259, 619-622 (1993) have reported that ceramide works as a second messenger of inflammatory cytokines, such as TNF-α and IL-1β, and activates arachidonic pathways, such as phospholipase A₂; and ceramide thus may be considered as an exacerbating factor in inflammatory disorders;
ceramide exacerbates the reduction of CD4⁺ T-cell accompanied by apoptosis and HIV infection of brain cells in patients infected with HIV, see e.g. Coline M. G. et al, Proc. Assoc. Am. Physicians 109, 146-153 (1997) and Wilt S. et al, Ann. Neurol. 37, 381-394 (1995);
in Begum N. et al, Eur. J. Biochem, 238, 214-220 (1996) and in Hotamsligil, G. S. et al, Science, 271, 665-668 (1996) it is reported that TNF-α may cause insulin restistance in type 2 diabetes mellitus as a trigger and obesity and that ceramide is involved in the downregulation of TNF-α;
in Beuttler B. et al, J. Cardiovasc. Pharmacol. 25, S1-S8 (1995) it is disclosed that ceramide triggers septicemia caused by lipopolysaccharide;
according to Schissel, S. L. et al in J. Clin. Invest., 1455-1464 (1996) the increase of ceramide activates sphingomyelinase in the aggregating reaction of LDL which triggers atherosclerosis lesions;
from Michael J. M. et al, Cancer Res., 57, 3600-3605 (1997), Bose R. et al, Cell, 82, 405-414 (1995), and Jaffrezou, J. P. et al, EMBO J., 15, 2417-2424 (1996) it is known that ceramide promotes apoptosis of cancer cells in radiotherapy and chemotherapy;
by Itoh M. et al, Clinical Cancer Res. 8,415-23 (2003) it is shown that ceramide regulation is involved in drug resistance of leukemia cells: a decrease of ceramide level is associated with the chemoresistant condition in leukemia.

Also ceramide-1-phosphate (Cer-1-P), which is produced from ceramide by the action of ceramide kinase, e.g. by phosphorylation of the hydroxyl group at position 1 of various ceramide derivatives, e.g. including N-acylated-, such as N-hexanoyl-, N-octanoyl-, N-palmitoyl-D-erythro-sphingosine, shows physiological activities, e.g.
Cer-1-P produced by ceramide kinase upon calcium stimulation regulates the release of neuronal transmitters from brain synapses, see e.g. Bajjalieh S. M. et al, J. Biol. Chem. 264, 14354-14360 (1989) and modulating the action of ceramide kinase is thus expected to be of value in the treatment of various neuronal disorders, e.g. including Alzheimer's disease;
Cer-1-P is believed to inhibit various normal ceramide activities, see e.g. Dressler K. A. et al, J. Biol. Chem. 265, 14917-14921 (1990), maybe through inhibition of acid sphingomyelinase as evidenced by Gomez-Munoz A. et al, J. Lipid Res. 45, 99-105 and thus Cer-1-P is expected to modulate, e.g. suppress, various disorders, e.g. inflammatory disorders, e.g. including chronic arthritis, HIV-infection, type 2 diabetes mellitus caused by insulin resistance as a trigger, obesity, septicemia and atherosclerosis; i.e. by activation of ceramide kinase it is believed that such diseases may be treated;
Cer-1-P is believed to act primarily inside the cell where it facilitates vesicle transport. It has been implicated in phagocytosis, and therefore could play an important role during inflammation processes, Hinkovska-Galcheva V. T. et al, J. Biol. Chem 273, 33203-9 (1998);
the mitogenic activity of exogenously added Cer-1-P has also been shown (Gomez-Munoz A. et al Biochem J 325, 435-40 (1997). Therefore, this sphingolipid metabolite may be relevant to cell proliferation disorders, including but not limited to cancer and psoriasis.
Cer-1-P has been reported to mediate cytokine-and calcium ionophore-induced arachidonic release, by Pettus, B. J. et al, J. Biol. Chem. 278, 38206-13 (2003), and this group further indicated that C-1-P may directly activate cytosolic PLA2 (Pettus, B. J. et al, J. Biol. Chem 279, 11320-6, 2004) and this further evidences the possible role of Cer-1-P in inflammatory disorders;
Cer-1-P levels could also be relevant to the pathophysiology (e.g. susceptibility to retinitis pigmentosa) of the visual system as suggested by Tuson M. et al, Am. J. Hum. Genet. 74:128-38 (2004).

We have now found a polynucleotide (gene) which encodes a ceramide kinase like protein.

In one aspect the present invention provides an isolated polynucleotide of sequence SEQ ID NO:1.

We also have found a polynucleotide of sequence SEQ ID NO:2 and a polynucleotide of sequence SEQ ID NO:3 which constitute 5′, or 3′, respectively, untranslated regions of a polynucleotide of sequence SEQ ID NO:1. The sum of polynucleotides of the sequences SEQ ID NO:1, SEQ ID NO:2 and SEQ ID NO:3 provides a 3203 bp cDNA of polynucleotide sequence SEQ ID NO:4.

In another aspect the present invention provides an isolated polynucleotide of sequence
SEQ ID NO:2,
SEQ ID NO:3, or
SEQ ID NO:4.

We also have found variants of a polynucleotide of sequence SEQ ID NO:1, e.g. of polynucleotide sequences SEQ ID NO:5, SEQ ID NO:6, SEQ ID NO:7, SEQ ID NO:8 and SEQ ID NO:9, which we have found to be splice variants of a poylnucleotide of sequence SEQ ID NO:1.

In another aspect the present invention provides an isolated polynucleotide which is a splice variant of a polynucleotide of sequence SEQ ID NO:1, such as an isolated polynucleotide of sequence
SEQ ID NO:5,
SEQ ID NO:6,
SEQ ID NO:7,
SEQ ID NO:8, or
SEQ ID NO:9.

Isolated polynucleotide means that the polynucleotide is isolated from its natural environment.

We have found that the gene or polynucleotide of sequence SEQ ID NO:1 provides the open reading frame (ORF) for encoding a ceramide kinase like protein, e.g. a splice variant of a polynucleotide of sequence SEQ ID NO:1 encodes a part of such protein. We have further estimated that a ceramide kinase like protein according to the present invention has the function of a ceramide kinase.

In another aspect the present invention provides a ceramide kinase like protein encoded by a polynucleotide of sequence
SEQ ID NO:1,
SEQ ID NO:5,
SEQ ID NO:6,
SEQ ID NO:7,
SEQ ID NO:8, or
SEQ ID NO:9,
   e.g. a ceramide kinase like protein encoded by a polynucleotide of SEQ ID NO:5, SEQ ID NO:6, SEQ ID NO:7, SEQ ID NO:8, and SEQ ID NO:9, each is a part of a ceramide kinase like protein encoded by a polynucleotide of sequence SEQ ID NO:1.

We have found that a polynucleotide of sequence
SEQ ID NO:1 encodes a polypeptide of sequence SEQ ID NO:10,
SEQ ID NO:5 encodes a polypeptide of sequence SEQ ID NO:11,
SEQ ID NO:6 encodes a polypeptide of sequence SEQ ID NO:12,
SEQ ID NO:7 encodes a polypeptide of sequence SEQ ID NO:13,
SEQ ID NO:8 encodes a polypeptide of sequence SEQ ID NO:14, and
SEQ ID NO:9 encodes a polypeptide of sequence SEQ ID NO:15.

In another aspect the present invention provides an isolated polypeptide of sequence
SEQ ID NO:10,
SEQ ID NO:11,
SEQ ID NO:12,
SEQ ID NO:13,
SEQ ID NO:14, or
SEQ ID NO:15.

A polynucleotide of the present invention is designated herein as “gene of (according to) the present invention”. A ceramide kinase like protein encoded by a gene of the present invention, which could phosphorylate ceramide itself or a metabolite of ceramide, is designated herein as “ceramide kinase like protein of (according to) the present invention”.

A gene of the present invention includes a nucleic acid sequence of SEQ ID NO:1, e.g. and allelic variants therof, and their complements; and splice variants thereof, e.g. such as polynucleotides of sequences SEQ ID NO:5, SEQ ID NO:6, SEQ ID NO:7, SEQ ID NO:8, or SEQ ID NO:9, e.g. including a polynucleotide that hybridizes to a nucleic acid sequence of a gene of the present invention, e.g. under stringent conditions. E. g. the nucleotide sequence of a gene of the present invention includes a sequence, which is different, e.g. as a result of the redundancy (degeneracy) of the genetic code, from the sequence of a gene of the present invention, but also encodes a ceramide kinase like protein of the present invention, e.g. a part thereof, e.g. having the same biological activity e.g. or encodes a ceramide kinase like protein of the present invention having an amino acid sequence which has at least 80%, e.g. 80% to 100%, such as 90%, e.g. 95%, e.g. 97%, e.g. 99% or 100%, identity; said identity being calculated by nₐ=xₐ−(xₐ. y), wherein nₐ is the number of amino acid alterations, xₐ is the total number of amino acids in said corresponding amino acid sequence, and y is percent identitiy divided by 100 and having the same biological activity; e.g. including a ceramide kinase like protein of the present invention produced with an allelic variant or a splice variant of a gene of the present invention. “Identity” is a measure of the identity of nucleotide sequences or amino acid sequences and may e.g. be calculated by techniques according to a method as conventional, e.g. by, e.g. commercially available, computer programs. “Stringent conditions” includes that hybridization will occur only if there is at least 80%, e.g. 90%, such as 95%, 97% or 99% identity between the nucleotide sequence of a gene of the present invention and the corresponding polynucleotide that hybridizes.

A ceramide kinase like protein of the present invention includes a polypeptide with an amino acid sequence encoded by a gene of the present invention, e.g. of SEQ ID NO:10, e.g. including a part of a ceramide kinase like protein, such as of polypeptides of sequence SEQ ID NO:11, SEQ ID NO:12 SEQ ID NO:13, SEQ ID NO:14 or SEQ ID NO:15, and including a polypeptide with an amino acid sequence which has at least 80% identity with an amino acid sequence of a polypeptide of the present invention.

A ceramide kinase like protein of the present invention may be in the form of the “mature” polypeptide, or may be part of a larger polypeptide, such as a fusion protein; e.g. it may be advantageous to include an additional amino acid sequence which contains secretory or leader sequences, pro-sequences, sequences which aid in purification such as multiple histidine residues, or an additional sequence for stability during recombinant production into a polypeptide of a ceramide kinase like protein of the present invention.

A ceramide kinase like protein of the present invention also includes a polypeptide fragment of a polypeptide of a ceramide kinase like protein of the present invention, e.g. such as encoded by a splice variant of a polynucleotide of the present invention. Such polypeptide fragment is meant to be a polypeptide having an amino acid sequence that entirely is the same in part, but not in all, of the amino acid sequence of a polypeptide of a ceramide kinase like protein of the present invention. Such polypeptide fragment may be “free-standing,” or may be part of a larger polypeptide of which such polypeptide fragment form a part or region, most preferably as a single continuous region. Preferably such polypeptide fragment retains the biological activity of the corresponding ceramide kinase of the present invention, at least in part.

A polypeptide of a ceramide kinase like protein of the present invention also includes variants of defined polypeptide (fragment) sequences of a ceramide kinase like protein of the present invention. Preferred variants are those that vary from the referents by conservative amino acid substitutions, e.g. variants wherein a residue is substituted by another of similar characteristics in the biological function.

“Polynucleotide”, if not otherwise specified herein, includes any polyribonucleotide or polydeoxyribonucleotide, which may be unmodified RNA or DNA, or modified RNA or DNA, including without limitation single and double stranded RNA, and RNA that is a mixture of single- and double-stranded regions. “Polypeptide”, if not otherwise specified herein, includes any peptide or protein comprising amino acids joined to each other by peptide bonds. Isolated polypeptide means that the polypeptide is isolated from its natural environment.

A ceramide kinase like protein of the present invention may be obtained analogously to a method as conventional, e.g. using standard cloning from a cDNA library derived from mRNA of brain cells comprising a gene of the present invention.

The polynucleotide encoding the ceramide kinase like protein of the present invention is obtained from a brain cDNA library. We have designed primers to cover the full putative open reading frame (ORF) identified in silico, and have used said primers in a Polymerase Chain Reaction protocol in order to amplify the ORF. We have obtained a fragment with the expected size and we performed cloning and sequencing which confirmed the expected sequence and further enabled us to detect various splice variants. By sequence comparison with a known ceramide kinase (accession number AB079066), we conclude that the ceramide kinase like protein of the present invention is a novel member of a Ceramide Kinase subfamily of the Diacylglycerol Kinase domain containing enzyme superfamily, e.g. because of homology considerations.

A gene of the present invention may be used for the recombinant production of a ceramide kinase like protein of the present invention. The polynucleotide of SEQ ID NO:1 includes the open reading frame (ORF) for encoding the sequence for the mature ceramide kinase like protein of the present invention, e.g. and optionally may contain other coding sequences, such as those encoding a leader or secretory sequence, a pre- or pro- or prepro- protein sequence, or other fusion peptide portions.

For example, a marker sequence which may facilitate purification of a fused polypeptide comprising a ceramide kinase like protein of the present invention can be encoded. The marker sequence may be an appropriate marker sequence, e.g. including conventional marker sequences, e.g. a hexa-histidine peptide, as provided in the pQE vector (Qiagen, Inc.) and described in Gentz et al, Proc Natl Acad Sci USA (1989)86:821-824, or an HA tag. Any polynucleotide according to the present invention may also contain non-coding 5′ and 3′ sequences (such as a polynucleotide of sequence SEQ ID NO:4), such as transcribed, non-translated sequences, splicing and polyadenylation signals, ribosome binding sites and sequences that stabilize mRNA.

A polynucleotide of the present invention may be introduced into a vector which may be used for genetically engineering (transforming, transfecting) a host cell, intended to express a polypeptide of the present invention.

In another aspect the present invention provides a vector comprising a polynucleotide of the present invention.

A vector comprising a gene of the present invention may be produced as appropriate, e.g. according to a method as conventional. An appropriate vector may be provided as appropriate, e.g. according to a method as conventional. A vector comprising a gene of the present invention may be useful to obtain an expression system which is able to produce a ceramide kinase like protein of the present invention encoded by a gene of the present invention recombinantly, e.g. in a host cell, such as in a compatible host cell. E. g. for recombinant production of a ceramide kinase like protein of the present invention a host cell may be genetically engineered, e.g. by use of a vector comprising a gene of the present invention, to incorporate into the host cell an expression system, e.g. or a part thereof, for expressing a ceramide kinase like protein of the present invention. Cell-free translation systems may also be used to produce a gene of the present invention, e.g. using RNAs derived from an DNA construct comprising a polynucleotide according to the present invention, e.g. analogously to a method as conventional.

In another aspect the present invention provides an expression system comprising a, e.g. pre-isolated, polynucleotide, e.g. DNA or RNA, of the present invention, wherein said expression system or part thereof is capable of producing a ceramide kinase like protein of the present invention when said expression system or part thereof is present in a compatible host cell.

In another aspect the present invention provides
a host cell comprising an expression system according to the present invention;
a process for producing a ceramide kinase like protein according to the present invention comprising culturing an isolated host cell comprising an expression system according to the present invention under conditions sufficient for the production of a ceramide kinase like protein of the present invention in the culture, and recovering, e.g. isolating, said ceramide kinase like protein of the present invention from the culture;
a process for the production of a recombinant host cell which produces a ceramide kinase like protein according to the present invention comprising transforming or transfecting a host cell with the expression system according to the present invention such that the host cell, under appropriate culture conditions, produces a ceramide kinase like protein according to the present invention; and
a recombinant host cell produced by transforming or transfecting a host cell with the expression system according to the present invention such that the host cell, under appropriate culture conditions, produces a ceramide kinase like protein according to the present invention.

Introduction of polynucleotides into host cells may be effected as appropriate, e.g. analogously to a method as conventional, e.g. according to Davis et al, BASIC METHODS IN MOLECULAR BIOLOGY (1986); Sambrook et al, MOLECULAR CLONING: A LABORATORY MANUAL, 2^{nd} Ed., Cold Spring Harbor Laboratory Press, Cold Spring Harbor, N. Y. (1989) such as calcium phosphate transfection, DEAE-dextran mediated transfection, transvection, microinjection, cationic lipid-mediated transfection, electroporation, transduction, scrape loading, ballistic introduction or infection. Host cells may be easily found. Examples of appropriate host cells include e.g. bacterial cells, such as streptococci, staphylococci, E. coli, Streptomyces and Bacillus subtilis cells; fungal cells, such as yeast cells and Aspergillus cells; insect cells such as Drosophila S2 and Spodoptera Sf9 cells; isolated animal cells such as CHO, COS, HeLa, C127, CCL39, 3T3, BHK, HEK 293 and Bowes melanoma cells; and plant cells.

Appropriate expression systems include e.g. chromosomal, episomal and virus-derived systems, e.g. vectors derived from bacterial plasmids, from bacteriophage, from transposons, from yeast episomes, from insertion elements, from yeast chromosomal elements, from viruses such as baculoviruses, papova viruses, such as SV40, vaccinia viruses, adenoviruses, fowl pox viruses, pseudorabies viruses and retroviruses, and vectors derived from combinations thereof, such as those derived from plasmid and bacteriophage genetic elements, e.g. cosmids and phagemids. An expression systems may contain control regions that regulate as well as engender expression. Generally, any system or vector suitable to maintain, propagate or express polynucleotides to produce a polypeptide in a host may be used. The appropriate nucleotide sequence may be inserted into an expression system as appropriate, e.g. analogously to a method as conventional, e.g. according to Sambrook et al, MOLECULAR CLONING A LABORATORY MANUAL (supra).

A ceramide kinase like protein according to the present invention may be recovered (isolated) and purified from recombinant cell cultures as appropriate, e.g. analogously to a method as conventional, e.g. including detergent extraction, ultracentrifugation, ammonium sulfate or ethanol precipitation, acid extraction, anion or cation exchange chromatography, phosphocellulose chromatography, hydrophobic interaction chromatography, affinity chromatography, hydroxylapatite chromatography, lectin chromatography, e.g. high performance liquid chromatography. If a ceramide kinase like protein according to the present invention is denatured during isolation and or purification, regeneration of the active conformation, e.g. refolding of a denaturated polypeptide, may be carried out as appropriate, e.g. analogously to a method as conventional.

A ceramide kinase like protein of the present invention provides in several aspects a target for a pharmaceutical against various diseases, e.g. including neuronal disorders, inflammatory disorders, e.g. psoriasis, HIV-infection, type 2 diabetes mellitus, e.g. caused by insulin restistance as a trigger, obesity, septicemia atherosclerosis, cancer, e.g. and retinitis pigmentosa. For example, by activation of a ceramide kinase it is reasonable that such diseases may be treated. Treatment includes treatment and prophylaxis. A ceramide kinase may also be useful in a method for detecting and/or quantifying the amount of ceramide in a sample. A splice variant of a gene of the present invention may be useful for screening for a full length ceramide kinase like protein, e.g. and as a diagnostic reagent. A splice variant such as the one reported in SEQ ID NO:6 would generate a truncated enzyme, bearing the catalytic domain but missing the C-terminal sequence which may present regulatory regions underlying substrate specificity or intracellular localization. Alternatively, such shorter form of a nucleotide of the present invention may be more suitable for assay development. A splice variant, e.g. of SEQ ID NO:7, would be even shorter and may lack kinase activity, and could act as a dominant negative mutant.

For example a gene (fragment, such as a splice variant) of the present invention or a ceramide kinase like protein (fragment) of the present invention may be used as a research reagent and as a tool for the discovery of treatments and diagnostics to animal and human diseases.

In another aspect the present invention provides the use of a polynucleotide, polypeptide or an antibody of the present invention as a diagnostic reagent.

Detection of a mutated form of a gene of the present invention associated with a dysfunction will provide a diagnostic tool, e.g. in a diagnostic assay, that may add to or define a diagnosis of a disease or susceptibility to a disease which results from under-expression, over-expression or altered expression of the corresponding gene or a mutant version therof. Individuals carrying mutations in the corresponding gene may be detected at the DNA level e.g. analogously to a method as conventional. This may be illustrated e.g. by the splice variant of SEQ ID NO:6, which bears two stretches of repeated DNA sequences (satellite DNA), flanking an additional exon.

Nucleic acids for diagnosis may be obtained from a subject's cells, such as from blood, urine, saliva, tissue biopsy or autopsy material. The genomic DNA may be used directly for detection or may be amplified enzymatcally by using PCR or other amplification techniques prior to analysis. RNA or cDNA may also be used in the analysis similarily. Deletions and insertions may be detected by a change in size of the amplified product in comparison to the normal genotype. Point mutations may be identified by hybridizing amplified DNA to labeled gene nucleotide sequences of the present invention. Perfectly matched sequences may be distinguished from mismatched duplexes by RNase digestion or by differences in melting temperatures. DNA sequence differences may also be detected by alterations in electrophoretic mobility of DNA fragments in gels, with or without denaturing agents, or by direct DNA sequencing, e.g. according to Myers et al, Science (1985) 230:1242. Sequence changes at specific locations may also be revealed by nuclease protection assays, such as RNase and S1 protection or the chemical cleavage method, e.g. according to Cotton et al, Proc Natl Acad Sci USA (1985) 85: 4397-4401. An array of oligonucleotides probes comprising the gene nucleotide sequence of the present invention or fragments thereof may be constructed to conduct efficient screening of e.g. genetic mutations. Array technology methods may e.g. be used to address a variety of questions in molecular genetics including gene expression, genetic linkage, and genetic variability, e.g. according to M. Chee et al, Science, Vol 274, pp 610-613, 1996.

A diagnostic assay offers a process for diagnosing or determining a susceptibility to diseases mediated by the action of a ceramide kinase like protein , e.g. including inflammatory disorders, e.g. chronic arthritis, psoriasis, e.g. including neuronal disorders, HIV-infection, type 2 diabetes mellitus caused by insulin restistance as a trigger, obesity, septicemia, artherosclerosis, cancer, e.g. and retinitis pigmentosa. Such diseases may be diagnosed e.g. analogously to a method as conventional, e.g. by methods comprising determining from a sample derived from a subject an abnormally decreased or increased level of
a ceramide kinase like protein of the present invention,
a secondary metabolite of such ceramide kinase like protein, such as ceramide-1-phosphate, or a related lipid metabolite-phosphate,
a gene mRNA of the present invention.

Decreased or increased expression levels can be determined at the RNA level, e.g. according to a method as conventional for the quantitation of polynucleotides, such as, for example, PCR, RT-PCR, RNase protection, Northern blotting and other hybridization methods. Assay techniques that may be used to determine levels of a protein, such as a ceramide kinase like protein of the present invention, or to detemine secondary metabolites of such ceramide kinase like protein in a sample derived from a host may be carried out as appropriate, e.g. analogously to a method as conventional. Such assay techniques include radioimmunoassays, competitive-binding assays, Western Blot analysis, ELISA and methods for detecting the amount of secondary metabolites, e.g. ceramide-1-phosphate, e.g. including fluorescent methods, mass spectrometry and chromatography.

In another aspect the present invention provides a diagnostic kit for a disease or susceptibility to a disease, such as inflammatory disorders, e.g. chronic arthritis, psoriasis, e.g. including neuronal disorders, HIV-infection, type 2 diabetes mellitus caused by insulin resistance as a trigger, obesity, septicemia, artherosclerosis, cancer, e.g. and retinitis pigmentosa, comprising as a main component
a) a gene (polynucleotide) of the present invention, e.g. including allelic variants thereof, or a fragment thereof; or a splice variant thereof, or
b) a nucleotide sequence complementary to that of (a), or
c) a ceramide kinase like protein of the present invention, or
d) an antibody against a ceramide kinase like protein of the present invention, e.g. any such kit, (a), (b), (c) or (d) may comprise a substantial component, e.g. including
   an appropriate environment of a sample,
   appropriate means to determine the effect of any of a), b), c) or d) in a sample to be tested.

A gene of the present invention may also be useful for chromosome identification. The sequence is specifically targeted to and can hybridize with a particular location on an individual human chromosome. The mapping of relevant sequences to chromosomes according to the present invention is an important first step in correlating those sequences with gene associated disease. Once a sequence has been mapped to a precise chromosomal location, the physical position of the sequence on the chromosome may be correlated with genetic map data. Corresponding data is e.g. disclosed e.g. in V. McKusick, Mendelian Inheritance in Man (available on line through Johns Hopkins University Welch Medical Library). The relationship between genes and diseases that have been mapped to the same chromosomal region may be identified through linkage analysis (coinheritance of physically adjacent genes). The differences in the cDNA or genomic sequence between affected and unaffected individuals may also be determined. If a mutation is observed in some or all of the affected individuals but not in any normal individuals, then the mutation is likely to be the causative agent of the disease.

A ceramide kinase like protein of the present invention, or fragment thereof, or cells expressing a polypetide according to the present invention can also be used as immunogens to produce antibodies immunospecific for the ceramide kinase polypeptide of the present invention. The term “immunospecific” means that the antibodies have substantially greater affinity for said polypeptide than their affinity for other related polypeptides. Antibodies generated against such polypeptide may e.g. be obtained by administering the polypeptide or an epitope-bearing fragment-analogue or cell to an animal, preferably a non-human, using routine protocols. For preparation of monoclonal antibodies, an appropriate technique which provides antibodies, e.g. produced by continuous cell line cultures, may be used, e.g. including the hybridoma technique (Kohler, G. and Milstein, C., Nature (1975) 256:495-497), the trioma technique, the human B-cell hybridoma technique (Kozbor et al, Immunology Today (1983) 4:72) and the EBV-hybridoma technique (Cole et al, MONOCLONAL ANTIBODIES AND CANCER THERAPY, pp. 77-96, Alan R. Liss, Inc., 1985). Techniques for the production of single chain antibodies (e.g. U.S. Pat. No. 4,946,778) can also be adapted to produce single chain antibodies to a polypeptide according to the present invention. Also, transgenic mice, or other organisms including other mammals, may be used to express humanized antibodies. Antibodies as described above may be used, e.g. in the isolation or in the identification of a clone expressing a polypeptide according to the present invention or for the purification of a polypeptide (fragment) according to the present invention by affinity chromatography.

In another aspect the present invention provides an antibody against a ceramide kinase like polypeptide of the present invention.

A ceramide kinase like protein of the present invention may be responsible for many biological functions, including many pathologies, e.g. such as described above. Accordingly, it is desirous to find compounds/drugs which either stimulate (agonists)
a ceramide kinase of the present invention, e.g. to produce secondary metabolites,
the expression of a gene of the present invention, e.g. to stimulate ceramide kinase expression,
   or which reduce or inhibit (antagonists)
the action of a ceramide kinase like protein of the present invention, e.g. to reduce or inhibit the production of secondary metabolites
expression of a gene of the present invention.

A ceramide kinase like protein of the present invention or functional mimetics thereof, e.g. according to Coligan et al, Current Protocols in Immunology 1 (2):Chapter 5 (1991), may thus be used to assess the binding of agonists or antagonists to a receptor part of the polypeptide of the ceramide kinase like protein of the present invention, e.g. in cells, cell-free preparations, chemical libraries, and natural product mixtures, e.g. in a screening assay. Such agonists and antagonists may be used in the treatment of diseases, such as inflammatory disorders, e.g. chronic arthritis, psoriasis, e.g. including neuronal disorders, HIV-infection, type 2 diabetes mellitus caused by insulin restistance as a trigger, obesity, septicemia, artherosclerosis, cancer, e.g. and retinitis pigmentosa.

Screening procedures may involve the production of appropriate cells in which ceramide kinase is expressed. Appropriate cells include cells e.g. from mammals, yeast, Drosophila. Cells expressing the ceramide kinase like protein of this invention (or cell membranes containing the expressed kinase) may be contacted with a candidate compound to observe binding, or stimulation or inhibition of a functional response.

A screening assay may be used to test a binding of a candidate compound wherein adherence to the cells bearing the receptor may be detected by means of a label directly or indirectly associated with the candidate compound or In an assay involving competition with a labeled competitor. Inhibitors of activation may be assayed in the presence and in the absence of a known agonist. A screening assay may comprise the steps of mixing a candidate compound with a solution containing a ceramide kinase like protein of the present invention to form a mixture, determining activity of said ceramide kinase like protein in the mixture, and comparing the activity of the mixture with the activity of a standard. A gene (cDNA) according to the present invention, a polypeptide of a ceramide kinase like protein according to the present invention and antibodies to such polypeptide of the present invention may also be used to provide a screening assay for detecting the effect of candidate compounds on the production of said gene (mRNA) and said polypeptide in cells. For example, an ELISA may be constructed for determining cell associated levels of said polypeptide, e.g. using monoclonal and polyclonal antibodies according to a method as conventional, and that ELISA may be used to discover agents (agonists or antagonists) which may increase or inhibit the production or the activity of said polypeptide from suitably manipulated cells or tissues. An assay for screening may be conducted, e.g. according to a method as conventional.

Examples of potential (ant)agonists of a gene of the present invention include antibodies or, in some cases, oligonucleotides or protein closely related to the ligand ((ant)agonist bound to a polypeptide of said gene) of said gene, e.g. a fragment of said ligand, or small molecules, which bind to the gene product of the present invention but do not elicit a response, or the product of a mutated form of the gene of the present invention so that the activity of the receptor is prevented.

Examples of potential (ant)agonists according to the present invention include compounds which bind to a polypeptide of a ceramide kinase like protein of the present invention, e.g. including oligopeptides, polypeptides, protein, antibodies, mimetics, small molecules, e.g. low molecular weight compounds (LMW's).

Thus in another aspect, the present invention provides a screening assay for identifying an agonist or an antagonist of a polypeptide of a ceramide kinase like protein of the present invention which assay comprises as a main component
a) a polypeptide of a ceramide kinase like protein of the present invention, or
b) a recombinant cell expressing a polypeptide of a), or
c) a cell membrane expressing a polypeptide according of a), or
d) an antibody to a polypeptide of a);
e.g. and means for a contact with a candidate compound; e.g. and means for determining the effect of the candidate compound on any of a), b), c) or d),
e.g. determining whether in the presence of the candidate compound there is a decrease or increase in the production and or the biological activity of a polypeptide of a);
e.g. by comparison of the activity of any of a), b), c) or d) in the presence and in the absence of the candidate compound;
and in another aspect

A method of identfying an agonist or antagonist, e.g. including ligands, receptors, antibodies or LMW's, which increases or decreases the production and/or the biological activity of a polypeptide of a ceramide kinase like protein of the present invention, which comprises
A) contacting
   a) a polypeptde of a ceramide kinase like protein of the present invention, or
   b) a recombinant cell expressing a polypeptide of a), or
   c) a cell membrane expressing a polypeptide of a), or
   d) an antibody to a polypeptide of a)
      with a candidate compound,
B) determining the effect of the candidate compound on any of a), b), c) or d);
   e.g. determining whether in the presence of the candidate compound there is a decrease or increase in the production and or the biological activity of a ceramide kinase of the present invention;
      e.g. by comparison of the activity of any of a), b), c) or d) in the presence and in the absence of the candidate compound,
C) choosing an agonist or antagonist determined in step B),
   e,g, choosing an appropriate candidate compound from which an agonist/antagonist effect is positively determined in step B).

It will be appreciated that in any such screening assay, a), b), c) or d) may comprise a substantial component. A candidate compound includes compound (libraries), from which the effect on any of a), b), c) or d) is unknown but may be expected. Compound (libraries) include compounds which are set out above as (ant)agonists to a polypeptide according to the present invention. An (ant)agonist is a candidate compound from which an effect on any of a), b), c) or d) has been found in a screening assay or in a method for identifying (ant)agonists as described above. An (ant)agonist may decrease or increase the production and or the biological activity of a polypeptide according to the present invention.

In another aspect the present invention provides an agonist or an antagonist, preferably an antagonist, of a polypeptide of a ceramide kinase like protein of the present invention, which is characterized in that said agonist or antagonist can be provided by a method of identfying an agonist or antagonist of the present invention.

An (ant)agonist of a polypeptide according to the present invention may be used in the treatment of diseases e.g. including neuronal disorders, inflammatory disorders, HIV-infection, type 2 diabetes mellitus, e.g. caused by insulin resistance as a trigger, obesity, septicemia arteriosclerosis, cancer, e.g. and retinitis pigmentosa. An (ant)agonist of a polypeptide according to the present invention may thus be useful as a pharmaceutical.

In another aspect the present invention provides an agonist or an antagonist of a polypeptide of a ceramide kinase like protein of the present invention for use as a pharmaceutical, e.g. in the treatment of diseases, such as inflammatory disorders, e.g. chronic arthritis, psoriasis, e.g. including neuronal disorders, HIV-infection, type 2 diabetes mellitus caused by insulin restistance as a trigger, obesity, septicemia, artheroscierosis, cancer, e.g. and retinitis pigmentosa

and, in another aspect

A soluble form of a ceramide kinase like protein of the present invention for use as a pharmaceutical, e.g. for the treatment of diseases wherein an (ant)agonist of the present invention is suitable.

An (ant)antagonist of a ceramide kinase like protein of the present invention may be administered in the form of a pharmaceutical composition.

In another aspect the present invention provides
A pharmaceutical composition comprising an agonist or an antagonist of the present invention as an active ingredient in combination with pharmaceutically acceptable excipient(s), which is characterized in that said antagonist or agonist can be provided by a method of identfying an agonist or antagonist of the present invention;
A pharmaceutical composition comprising a soluble form of a ceramide kinase like protein of the present invention as an active ingredient in combination with pharmaceutically acceptable excipient(s).

Such pharmaceutical composition may be produced as appropriate, e.g. according, e.g. analogously, to a method as conventional, e.g. by mixing an (ant)agonist provided by the method steps A), B) and C) with excipients, e.g. and further processing the mixture obtained, to obtain a pharmaceutical composition for appropriate administration.

In a further aspect the present invention provides a method of treating abnormal conditions mediated by a ceramide kinase like protein,
e.g. a method of treatment of inflammatory disorders, e.g. chronic arthritis, psoriasis, e.g. including neuronal disorders, HIV-infection, type 2 diabetes mellitus caused by insulin restistance as a trigger, obesity, septicemia, artheroscierosis, cancer, e.g. and retinitis pigmentosa,
comprising administering a therapeutically effective amount of an agonist or antagonist of the present invention, e.g. which can be provided by the method steps A), B) or C) as described above, e.g. in combination with pharmaceutically acceptable excipient(s);
e.g. or administering a therapeutically amount of a soluble form of a polypeptide of a ceramide kinase like protein of the present invention,
e.g. in combination with pharmaceutically acceptable excipients, e.g. in the form of a pharmaceutical composition;
   to a subject in need of such treatment.

A phamaceutical composition of the present invention may be administered by any conventional route, for example enterally, e.g. including nasal, buccal, rectal, oral, administration; parenterally, e.g. including intravenous, intramuscular, subcutanous administration; or topically; e.g. including epicutaneous, intranasal, intratracheal administration, e.g. in form of coated or uncoated tablets, capsules, (injectable) solutions, solid solutions, suspensions, dispersions, solid dispersions; e.g. in the form of ampoules, vials, in the form of creams, gels, pastes, inhaler powder, foams, tinctures, lip sticks, drops, sprays, or in the form of suppositories.

Preferred forms of systemic administration of a pharmaceutical composition of the present invention include injection, typically by intravenous injection. Other injection routes, such as subcutaneous, intramuscular, or intraperitoneal, may be used. Alternative means for systemic administration include transmucosal and transdermal administration, e.g. using penetrants such as bile salts or fusidic acids or other detergents. In addition, if properly formulated in enteric or encapsulated formulations, oral administration may also be possible. Administration of a composition according to the present invention may also be topical and/or localized.

For such treatment, the appropriate dosage will, of course, vary depending upon, for example, the chemical nature and the pharmakokinetic data of a compound of the present invention employed, the individual host, the mode of administration, the nature and severity of the conditions being treated and whether a polypeptide of a ceramide kinase like protein of the present invention is used as an active ingredient, or an (ant)agonist of the present invention. However, in general, for satisfactory results in larger mammals, for example humans, an indicated daily dosage is In the range from about 0.1 mg to about 1500 mg (ca 10 μg/kg to 20 mg/kg of body weight). Variations in the needed dosage, however, maybe expected in view of the variety of compounds available and the differing efficiencies of various routes of administration. For example, oral administration would be expected to require higher dosages than administration by intravenous injection. Variations in these dosage levels may be adjusted as appropriate, e.g. according to standard empirical routine for optimization.

Polypeptides used in treatment may also be generated endogenously in a subject, e.g. in need of such treatment, in treatment modalities often referred to as “gene therapy”, e.g. as described above. Thus, for example, cells from a subject may be engineered ex vivo with a polynucleotide, such as a DNA or RNA, to encode a polypeptide according to the present invention, e.g. by use of a retroviral plasmid vector. Engineered cells may be introduced into a subject in need of such treatment.

In the following examples all temperatures are in degree Centigrade and are uncorrected.

Abbreviations Used

PCR Polymerase Chain reaction
ORF Open Reading Frame

### EXAMPLE 1

Search for a cDNA clone having homology to human ceramide kinase is based on partial information from two clones of the NCBI database, BC020465 and XM_{—}087153. Their assembly is expected to allow to produce a polynucleotide in which a putative ORF coding for a Ceramide Kinase like protein is detected. The sequence is fully recovered from human chromosome 2.

### EXAMPLE 2

Homology

Sequence homology is addressed using the BLAST algorithm and the GAP GCG software. The ORF claimed herein was determined to have 45.662% homology at the nucleotide level, and 33.871% similarity (24.798% homology) at the amino acid level with ceramide kinase (cf Accession number AB079066, and J Biol Chem, 2002, vol 277, 23294-300). Like ceramide kinase, the decuced amino acid sequence of the ORF (gene) of the present invention displays a DAGK like domain. Interestingly, a stretch of protein sequence well conserved in the ceramide kinase of various species and not found in the sphingosine kinase consensus sequence, is also present in the protein sequence deduced from the ORF of the present invention. It is therefore reasonably expected that the protein encoded by the ORF of the present invention is “ceramide kinase like protein”.

BLAST searches also indicate that the ORF of the present invention likely has orthologs in rat and mouse.

### EXAMPLE 3

Cloning

The following oligonucleotides are designed to amplify the ORF: 5′ ccagcctgcgactccgccatgccc 3′ and 5′ ttactttggaatcatttcttccatgcttcctcc 3′ A 25 μl PCR recation is run on 2,5 μl human brain cDNA (Marathon brain cDNA library, Clontech), 400mM of each of the above mentioned oligonucleotides, Advantage 2 PCR polymerase (Clontech) using the following amplification parameters: 94° for 5 minutes, 5 cycles of 94° for 30 seconds, 65° for 30 seconds, 68° 3 minutes for 30 seconds, 25 cycles of 94° for 30 seconds, 58° for 30 seconds, 68° for 3 minutes 30 seconds, followed by a final extension step at 68° for 5 minutes. This is made on a MJ PTC-200 thermal cycler. Results are analyzed on a 1% agarose gel (SeaPlaque GTG agarose, Cambrex). Three discrete bands are observed in the 1,6-2,4 kb range. They are cut and their DNA content extracted (MinElute gel extraction kit, Qiagen). DNA is either cloned directly in pCR4-TOPO (Invitrogen), after A addition (A-addition kit, Qiagen), or cloned in pENTR/SD/D-TOPO (Invitrogen) after a subsequent PCR designed to add a CACC sequence at the 5′ end of the DNA fragment. This additional PCR is done with the following primers: 5′ caccatgccctggaggaggcgcagg 3′ and 5′ ttactttggaatcatttcttccatgcttcctcc 3′ according to the next cycling parameters: 94° for 2 minutes, 5 times: 94° for 10 seconds, 72° for 4 minutes, 5 times: 94° for 10 seconds, 69° for 4 minutes, 23 times: 94° for 10 seconds, 67° for 4 minutes, followed by a final extension step of 5 minutes at 72°. Amplified DNA is resolved on 1% agarose gel and further purified as described above.

TOPO cloning reactions are undertaken according to the manufacturer's instructions (Invitrogen). 2 μl of each TOPO reaction mixture is used to transform 50 μl TOP 10 E.coli according to the manufacturer's instructions (Invitrogen). Recombinant clones are selected on LB+kanamycin. Plasmids are prepared according to Qiagen's miniprep procedure.

### EXAMPLE 4

Expression

Expression survey is performed by PCR using a human RAPID-SCAN Gene Expression panel (OriGene) and the following oligonucleotides: 5′ acattgcacattataatggggcatgtacagctggtcg 3′ and 5′ gttactttggaatcatttcttccatgcttcctcc 3′ with the PCR program #2 stated above. Expression is found to be tissue specific with strong amounts in adrenal gland, kidney, testis and lung, moderate amount in fetal liver, fetal brain, brain, spleen, prostate, bone marrow and stomach, little amount in muscle, thyroid, pancreas and PBL, and no detectable level in skin, small intestine, heart, colon, placenta, saliva, ovary and uterus. Other PCR-based experiments have shown that the Ceramide kinase like mRNA is also expressed in the trachea and the spinal cord. Detection of radiolabelled polypeptide after in vitro translation of tagged fusion proteins may be done by Western blot.

### EXAMPLE 5

Subcellular fractionation

Upon expression of the ceramide kinase like protein with a C-terminal flag tag followed by cell fractionation, the protein may be recovered from the cytosol as well as with the perimembrane fraction and also as Triton X-100 insoluble material.

### EXAMPLE 6

Subcellular localization

Using GFP tagged recombinant proteins expressed in COS-1 cells, the ceramide kinase like protein is found in the cytoplasm and in the nucleus, where in particular, it could also localize to nucleoli.

### EXAMPLE 7

Using recombinant overexpressed protein in COS-1 cells, the ceramide kinase like protein is found to contain phosphate molecules, primarily on threonine residues.

### EXAMPLE 8

Assay of various substrates, e.g. diacylglycerol, sphingosine, C2-, C6-, C8-, C16-, C18-ceramides, do not reveal activity of the ceramide kinase like protein. Also within COS-1 cells transiently expressing the ceramide kinase like protein and pulse labelled with ³²P, no specific phospholipids can be detected. It may well be that the ceramide kinase like protein of the present invention needs particular conditions to display activity such as, but not limited to, the assistance of a cofactor.

1. An isolated polynucleotide of sequence SEQ ID No:4.

2. An isolated polynucleotide according to claim 1, which is of sequence SEQ ID NO: 1.

3. An isolated polynucleotide according to claim 1, which is of sequence SEQ ID NO:2 or SEQ ID NO:3.

4. An isolated polynucleotide according to claim 1, which is of sequence
SEQ ID NO:5,
SEQ ID NO:6,
SEQ ID NO:7,
SEQ ID NO:8, or
SEQ ID NO:9.

5. An isolated polynucleotide of any of claim 1 encoding a ceramide kinase like protein.

6. An isolated polynucleotide, which is of sequence
SEQ ID NO:10,
SEQ ID NO:11,
SEQ ID NO:12,
SEQ ID NO:13,
SEQ ID NO:14, or
SEQ ID NO:15.

7. An isolated ceramide kinase like protein encoded by a polynucleotide of claim 1.

8. A Vector comprising a polynucleotide of claim 1.

9. An expression system comprising a polynucleotide of claim 1, wherein said expression system or part thereof is capable of producing a ceramide kinase like protein, when said expression system or part thereof is present in a compatible host cell.

10. A host cell comprising an expression system according to claim 9.

11. An antibody against a ceramide kinase like polypeptide according to claim 6.

12. Use of a polynucleotide of claim 1 as a diagnostic reagent.

13. A diagnostic kit for a disease or susceptibility to a disease comprising as a main component
a) a polynucleotide according to claim 1,
b) a nucleotide sequence complementary to that of a),
c) a ceramide kinase like protein, or
d) an antibody against a ceramide kinase like protein.

14. A method of identifying an agonist or antagonist which increases or decreases the production and/or the biological activity of a polypeptide of a ceramide kinase like protein according to claim 6, which method comprises
A) contacting
a) a polypeptide of a ceramide kinase like protein according to claim 6,
b) a recombinant cell expressing a polypeptide of a),
c) a cell membrane expressing apolypeptide of a), or
d) an antibody against a polypeptide of a) with a candidate compound,
B) determining the effect of the candidate compound on any of a), b), c) or d);
C) choosing an agonist or antagonist determined in step B.

15. An agonist or an antagonist of a polypeptide of a ceramide kinase like protein according to claim 6, which is characterized in that said agonist or antagonist can be provided by a method of claim 14.

16. An agonist or an antagonist of claim 15 for use as a pharmaceutical.

17. A method of treating abnormal conditions mediated by a ceramide kinase like protein comprising administering a therapeutically effective amount of an agonist or antagonist of claim 15 to a subject in need to such treatment.


SEQUENCE LISTING (15 sequences)

SEQ ID NO: 1 - DNA, 1599 bp, Homo sapiens
atgccctggaggaggcgcaggaaccgggtgagtgccctggagggcggccgggaggaagaggcgcccccggaggctgccgctgtgcctccggcgctgttaacgtccccgcagcagacggaggcggcggccgagcggattctgctccggggcatcttcgagatcgggagggacagttgtgacgtggtgctgagcgagcgagcactgcggtggcggcccattcagcccgagcgcccggcgggtgattctaagtatgacttgctatgtaaagaagaatttattgaactcaaagacatattctctgtgaaactgaaacggcgttgttctgttaaacagcagagaagtggtactttattaggtatcacactcttcatctgcttgaaaaaggaacaaaataaactaaagaattctacacttgatcttattaatttaagtgaagaccactgtgacatatggtttagacagttcaagaaaatattggcaggctttccaaacagaccgaagtcattaaaaatactccttaacccccaaagtcacaaaaaagaagctacccaggtttattatgagaaggttgaacctctgttgaagcttgcaggaataaaaactgatgtaacaataatggaatatgaagggcacgctctgtcactgcttaaggaatgtgaactccagggatttgatggtgttgtctgtgttggtggagatggatctgctagcgaagtagcccatgctttgcttctgagagctcagaagaatgctgggatggaaacagaccgaatcctgactcctgtcagagcacagcttccacttggcttaataccagcaggatctaccaatgtattggcacattctcttcatggagttcctcatgtgataactgcaacattgcacattataatggggcatgtacagctggtcgacgtctgcaccttcagcaccgctggcaagcttcttcgctttgggttctcagccatgtttggctttggtggaagaactttggctctggcagaaaaatatcgatggatgtcccctaaccaacggagagattttgctgttgttaaggcactggcaaaacttaaggcagaagactgtgaaatatcatttttaccatttaacagctctgatgatgtgcaagaaaggagggcacagggatctcccaaatctgactgtaatgatcaatggcaaatgatccagggtcagttcttgaatgtcagcattatggcaattccttgcctgtgttcagtggcacctagaggcttggcacctaataccagattaaataatggaagtatggctcttataattgcccgaaacacttctcggccagaatttataaaacacctgaaaagatatgccagtgtaaaaaatcagttcaattttccatttgttgagacttacactgttgaggaagtaaaagttcatccaaggaataatactggtggatataatccagaggaggaggaggatgaaactgcttcagaaaattgtttcccttggaatgtagatggtgatttaatggaagttgcatcagaggtccatattagattgcatccaagacttatcagtctttatggaggaagcatggaagaaatgattccaaagtaa

SEQ ID NO: 2 - DNA, 18 bp, Homo sapiens
ccagcctgcgactccgcc

SEQ ID NO: 3 - DNA, 1586 bp, Homo sapiens
catgtaattgtttctaaaagaaatgtacaaactagaattttaatataaagaaatgtggccatgctttaaacctgtggtccccaccctttttgggaccagtttcatggaagacaatttttctgtggatgggggaatggtttcaggatgaactgttccacctcagatcatcgggcattagattctcttgaggaacatggcactctagatccctcacataccagttcaccatatggttcgcacttctatgagaatctaatgctgctgctgatctgacagggggaggaactcagtaatgctccattgctctaacctcctgctgtgtggccaaacggatatcctaatagaaattgatgttactaacaattagcctctagggctttaaaagtcaagatgaaatatcaaagtcaagatgaaatatcaattcaaattctgtttttagatatcaattcaaattcttttttttacaatgtgtttattggacacacaaaaaaactttgcaaccatcataatacatcaatatttaacctagataattctgaaataatttggattctttcattttcaggatttgagctcatcaattatgttaaatttcctatattctgttacaaatataatacagatttcataagtctgccttgattcactgctccctaatccagagcagttaaatttcaaaatcacatagttatatagcctacctgccaaaaaagaataacccatccctgtcttcttaaagtggtagggttttaaaattcatgatacaagtaacaaagtaagttgataacaaatactattttgaactggacaaaatctagcccaatatgtaattgtgttccccttgtatctgagaggcctgtgttggacaaccatagtactctgctttcactgtgctcaaataacctgaaatatatagttgtttctgagtagttctgagtaggaaagaaccaaatgtaaatttccttagagaccatcctggagaagacagttctaaatcggggcttgatttttcatctatgatcctagatgttctcctgaaagaatgactagcctttttataacgatacaaagttttttaaaaatacataaatgtcataaagtttattcctgtgtatacacactctgggttcaggaggttttgtgcttacatttcactactcaaatgccttcgcaaatttcagaacattcctcaccatcactgcaggcctgtgaacagataactttcatcaattaggatggctggctagtggcagggacttcatataaagaaatctttcccagaattgtttttccacattgccttctaatttgatcactccctataacagatgttcaaaattcggccacagctctcttatgaagacagagtcagataaaatgatattctgcaattatctagatcataagcataaaataaatattacaaaaatgattgtatttaaagaaattttttaaaatccagaaagtcatttaaaatagaacctcatatagtatgaacaaactataaaaatattttacatttcaaaaaaaaaaaaaaaaaaaaaaagcggccgctgaattctagacctgcccgggcggccgctcga

SEQ ID NO: 4 - DNA, 3203 bp, Homo sapiens
ccagcctgcgactccgccatgccctggaggaggcgcaggaaccgggtgagtgccctggagggcggccgggaggaagaggcgcccccggaggctgccgctgtgcctccggcgctgttaacgtccccgcagcagacggaggcggcggccgagcggattctgctccggggcatcttcgagatcgggagggacagttgtgacgtggtgctgagcgagcgagcactgcggtggcggcccattcagcccgagcgcccggcgggtgattctaagtatgacttgctatgtaaagaagaatttattgaactcaaagacatattctctgtgaaactgaaacggcgttgttctgttaaacagcagagaagtggtactttattaggtatcacactcttcatctgcttgaaaaaggaacaaaataaactaaagaattctacacttgatcttattaatttaagtgaagaccactgtgacatatggtttagacagttcaagaaaatattggcaggctttccaaacagaccgaagtcattaaaaatactccttaacccccaaagtcacaaaaaagaagctacccaggtttattatgagaaggttgaacctctgttgaagcttgcaggaataaaaactgatgtaacaataatggaatatgaagggcacgctctgtcactgcttaaggaatgtgaactccagggatttgatggtgttgtctgtgttggtggagatggatctgctagcgaagtagcccatgctttgcttctgagagctcagaagaatgctgggatggaaacagaccgaatcctgactcctgtcagagcacagcttccacttggcttaataccagcaggatctaccaatgtattggcacattctcttcatggagttcctcatgtgataactgcaacattgcacattataatggggcatgtacagctggtcgacgtctgcaccttcagcaccgctggcaagcttcttcgctttgggttctcagccatgtttggctttggtggaagaactttggctctggcagaaaaatatcgatggatgtcccctaaccaacggagagattttgctgttgttaaggcactggcaaaacttaaggcagaagactgtgaaatatcatttttaccatttaacagctctgatgatgtgcaagaaaggagggcacagggatctcccaaatctgactgtaatgatcaatggcaaatgatccagggtcagttcttgaatgtcagcattatggcaattccttgcctgtgttcagtggcacctagaggcttggcacctaataccagattaaataatggaagtatggctcttataattgcccgaaacacttctcggccagaatttataaaacacctgaaaagatatgccagtgtaaaaaatcagttcaattttccatttgttgagacttacactgttgaggaagtaaaagttcatccaaggaataatactggtggatataatccagaggaggaggaggatgaaactgcttcagaaaattgtttcccttggaatgtagatggtgatttaatggaagttgcatcagaggtccatattagattgcatccaagacttatcagtctttatggaggaagcatggaagaaatgattccaaagtaacatgtaattgtttctaaaagaaatgtacaaactagaattttaatataaagaaatgtggccatgctttaaacctgtggtccccaccctttttgggaccagtttcatggaagacaatttttctgtggatgggggaatggtttcaggatgaactgttccacctcagatcatcgggcattagattctcttgaggaacatggcactctagatccctcacataccagttcaccatatggttcgcacttctatgagaatctaatgctgctgctgatctgacagggggaggaactcagtaatgctccattgctctaacctcctgctgtgtggccaaacggatatcctaatagaaattgatgttactaacaattagcctctagggctttaaaagtcaagatgaaatatcaaagtcaagatgaaatatcaattcaaattctgtttttagatatcaattcaaattcttttttttacaatgtgtttattggacacacaaaaaaactttgcaaccatcataatacatcaatatttaacctagataattctgaaataatttggattctttcattttcaggatttgagctcatcaattatgttaaatttcctatattctgttacaaatataatacagatttcataagtctgccttgattcactgctccctaatccagagcagttaaatttcaaaatcacatagttatatagcctacctgccaaaaaagaataacccatccctgtcttcttaaagtggtagggttttaaaattcatgatacaagtaacaaagtaagttgataacaaatactattttgaactggacaaaatctagcccaatatgtaattgtgttccccttgtatctgagaggcctgtgttggacaaccatagtactctgctttcactgtgctcaaataacctgaaatatatagttgtttctgagtagttctgagtaggaaagaaccaaatgtaaatttccttagagaccatcctggagaagacagttctaaatcggggcttgatttttcatctatgatcctagatgttctcctgaaagaatgactagcctttttataacgatacaaagttttttaaaaatacataaatgtcataaagtttattcctgtgtatacacactctgggttcaggaggttttgtgcttacatttcactactcaaatgccttcgcaaatttcagaacattcctcaccatcactgcaggcctgtgaacagataactttcatcaattaggatggctggctagtggcagggacttcatataaagaaatctttcccagaattgtttttccacattgccttctaatttgatcactccctataacagatgttcaaaattcggccacagctctcttatgaagacagagtcagataaaatgatattctgcaattatctagatcataagcataaaataaatattacaaaaatgattgtatttaaagaaattttttaaaatccagaaagtcatttaaaatagaacctcatatagtatgaacaaactataaaaatattttacatttcaaaaaaaaaaaaaaaaaaaaaaagcggccgctgaattctagacctgcccgggcggccgctcga

SEQ ID NO: 5 - DNA, 1376 bp, Homo sapiens
cgcccgggcaggtctagaattcagcggccgctgaattctagggggaggaggggagaacggggagcgcacagcctggacgcgtgcgcaggcgtcaggcgcatagacctgctagcccctcagctagcggccccgcccgcgcttagcatcactaactgggctatataacctgagcgcccgcgcggccacgacacgaggaattcgcccacgcaggaggcgcggcgtccggaggccccagggttatgagactatcactgctcaggacctactaacaacaaaggtgattctaagtatgacttgctatgtaaagaagaatttattgaactcaaagacatattctctgtgaaactgaaacggcgttgttctgttaaacagcagagaagtggtactttattaggtatcacactcttcatctgcttgaaaaaggaacaaaataaactaaagaattctacacttgatcttattaatttaagtgaagaccactgtgacatatggtttagacagttcaagaaaatattggcaggctttccaaacagaccgaagtcattaaaaatactccttaacccccaaagtcacaaaaaagaagctacccaggtttattatgagaaggttgaacctctgttgaagcttgcaggaataaaaactgatgtaacaataatggaatatgaagggcacgctctgtcactgcttaaggaatgtgaactccagggatttgatggtgttgtctgtgttggtggagatggatctgctagcgaagtagcccatgctttgcttctgagagctcagaagaatgctgggatggaaacagaccgaatcctgactcctgtcagagcacagcttccacttggcttaataccagcaggatctaccaatgtattggcacattctcttcatggagttcctcatgtgataactgcaacattgcacattataatggggcatgtacagctggtcgacgtctgcaccttcagcaccgctggcaagcttcttcgctttgggttctcagccatgtttggctttggtggaagaactttggctctggcagaaaaatatcgatggatgtcccctaaccaacggagagattttgctgttgttaaggcactggcaaaacttaaggcagaagactgtgaaatatcatttttaccatttaacagctctgatgatgtgcaagaaaggagggcacagggatctcccaaatctgactgtaatgatcaatggcaaatgatccagggtcagttcttgaatgtcagcattatggcaattccttgcctgtgttcagtggcacctagaggcttggcacctaataccagattaaataatggaagtatggctcttataattgcccgaaacacttctcggccagaatttataaaacacct

SEQ ID NO: 6 - DNA, 2405 bp, Homo sapiens
atgccctggaggaggcgcaggaaccgggtgagtgccctggagggcggccgggaggaagaggcgcccccggaggctgccgctgtgcctccggcgctgttaacgtccccgcagcagacggaggcggcggccgagcggattctgctccggggcatcttcgagatcgggagggacagttgtgacgtggtgctgagcgagcgagcactgcggtggcggcccattcagcccgagcgcccggcgggtgattctaagtatgacttgctatgtaaagaagaatttattgaactcaaagacatattctctgtgaaactgaaacggcgttgttctgttaaacagcagagaagtggtactttattaggtatcacactcttcatctgcttgaaaaaggaacaaaataaactaaagaattctacacttgatcttattaatttaagtgaagaccactgtgacatatggtttagacagttcaagaaaatattggcaggctttccaaacagaccgaagtcattaaaaatactccttaacccccaaagtcacaaaaaagaagctacccaggtttattatgagaaggttgaacctctgttgaagcttgcaggaataaaaactgatgtaacaataatggaatatgaagagcacgctctgtcactgcttaaggaatgtgaactccagggatttgatggtgttgtctgtgttggtggagatggatctgctagcgaagtagcccatgctttgcttctgagagctcagaagaatgctgggatggaaacagaccgaatcctgactcctgtcagagcacagcttccacttggcttaataccagcaggatctaccaatgtattggcacattctcttcatggagttcctcatgtgataactgcaacattgcacattataatggggcatgtacagctggtcgacgtctgcaccttcagcaccgctggcaagcttcttcgctttgggttctcagccatgtttggctttggtggaagaactttggctctggcagaaaaatatcgatggatgtcccctaaccaacggagagattttgctgttgttaaggcactggcaaaacttaaggcagaagactgtgaaatatcatttttaccatttaacagctctgatgatgtgcaagaaaggtaggtaaaaacccaatgtaatacagataacctgtatctttaaagcataaaacattctgacaaacaattgatcagacttaggatctttggttatttttctaacatagtagtaaagaatatagaatcacagactcttacaggtcctagtaagcaaacaactactcactcagcctttttctctttttccttccttctttcctcccttccttccccctgtctctccctccctccttccgttctctttttcccttcccttcccctttctttcctttcctttgtttttttccctttctttccttttccctttcccttttcttttgagtgcctactatatgccaggcactttgaatacaacaatgcaccaaaaaattaacaaaacacacagttttgtccctcgtgataattttggtctagtgtgtaatacaaatattaaagccatattaaagaagggttatagtctttaaagaaatagtacattattattccttataatactagtttacctttaaatgaatcaacacattatgttggcagcaacaaaattgtacacgtttttacttactatctaattgaagaaagtatttaacatatataatgatgagttctctaattttctcatatttaatagatttgtgtgtgtgtgtgtgtgtgtctgtgtgtctgtgcgcgcgttatctgttttatgtctttgttttgttttctttcttttaggagggcacagggatctcccaaatctggtgagtcgaaaagtatctccttgagaactgtgccaggttctgttgacattttgttctggtgagggaaaataaccaatcactttcattacctttagactgtaatgatcaatggcaaatgatccagggtcagttcttgaatgtcagcattatggcaattccttgcctgtgttcagtggcacctagaggcttggcacctaataccagattaaataatggaagtatggctcttataattgcccgaaacacttctcggccagaatttataaaacacctgaaaagatatgccagtgtaaaaaatcagttcaattttccatttgttgagacttacactgttgaggaagtaaaagttcatccaaggaataatactggtggatataatccagaggaggaggaggatgaaactgcttcagaaaattgtttcccttggaatgtagatggtgatttaatggaagttgcatcagaggtccatattagattgcatccaagacttatcagtctttatggaggaagcatggaagaaatgattccaaagtaa

SEQ ID NO: 7 - DNA, 1534 bp, Homo sapiens
atgccctggaggaggcgcaggaaccgggtgagtgccctggagggcggccgggaggaagaggcgcccccggaggctgccgctgtgcctccggcgctgttaacgtccccgcagcagacggaggcggcggccgagcggattctgctccggggcatcttcgagatcgggagggacagttgtgacgtggtgctgagcgagcgagcactgcggtggcggcccattcagcccgagcgcccggcgggtgattctaagtatgacttgctatgtaaagaagaatttattgaactcaaagacatattctctgtgaaactgaaacggcgttgttctgttaaacagcagagaagtggtactttattaggtatcacactcttcatctgcttgaaaaaggaacaaaataaactaaagaattctacacttgatcttattaatttaagtgaagaccactgtgacatatggtttagacagttcaagaaaatattggcaggctttccaaacagaccgaagtcattaaaaatactccttaacccccaaagtcgcaaaaaagaagctacccaggtttattatgagaaggttgaacctctgttgaagcttgcaggaataaaaactgatgtaacaatgttgtctgtgttggtggagatggatctgctagcgaagtagcccatgctttgcttctgagagctcagaagaatgctgggatggaaacagaccgaatcctgactcctgtcagagcacagcttccacttggcttaataccagcaggatctaccaatgtattggcacattctcttcatggagttcctcatgtgataactgcaacattgcacattataatggggcatgtacagctggtcgacgtctgcaccttcagcaccgctggcaagcttcttcgctttgggttctcagccatgtttggctttggtggaagaactttggctctggcagaaaaatatcgatggatgtcccctaaccaacggagagattttgctgttgttaaggcactggcaaaacttaaggcagaagactgtgaaatatcatttttaccatttaacagctctgatgatgkgcaagaaaggagggcacagggatctcccaaatctgactgtaatgatcaatggcaaatgatccagggtcagttcttgaatgtcagcattatggcaattccttgcctgtgttcagtggcacctagaggcttggcacctaataccagattaaataatggaagtatggctcttataattgcccgaaacacttctcggccagaatttataaaacacctgaaaagatatgccagtgtaaaaaatcagttcaattttccatttgttgagacttacactgttgaggaagtaaaagttcatccaaggaataatactggtggatataatccagaggaggaggaggatgaaactgcttcagaaaattgtttcccttggaatgtagatggtgatttaatggaagttgcatcagaggtccatattagattgcatccaagacttatcagtctttatggaggaagcatggaagaaatgattgcaaagaa

SEQ ID NO: 8 - DNA, 1393 bp, Homo sapiens
ccagcctgcgactccgccatgccctggaggaggcgcaggaaccgggtgagtgccctggagggcggccgggaggaagaggcgcccccggaggctgccgctgtgcctccggcgctgttaacgtccccgcagcagacggaggcggcggccgagcggattctgctccggggcatctttgagatcgggagggacagttgtgacgtggtgctgagcgagcgagcactgcggtggcggcccattcagcccgagcgcccggcgggtgattctaagtatgacttgctatgtaaagaagaatttattgaactcaaagacatattctctgtgaaactgaaacggcgttgttctgttaaacagcagagaagtggtactttattaggtatcacactcttcatctgcttgaaaaaggaccaaaataaactaaagattctacacttgatcttattaattaagtgaagacccactgtgacatatggttagacagttcaagaaaatattggcagtgttgtctgtgttggtggagatggatctgctagcgaagtagcccatgctttgcttctgagagctcagaagaatgctgggatggaaacagaccgaatcctgactcctgtcagagcacagcttccacttggcttaataccagcaggatctaccaatgtattggcacattctcttcatggagttcctcatgtgataactgcaacattgcacattataatggggcatgtacagctggtcgacgtctgcaccttcagcaccgctggcaagcttcttcgctttgggttctcagccatgtttggctttggtggaagaactttggctctggcagaaaaatatcgatggatgtcccctaaccaacggagagattttgctgttgttaaggcactggcaaaacttaaggcagaagactgtgaaatatcatttttaccatttaacagctctgatgatgtgcaagaaagactgtaatgatcaatggcaaatgatccagggtcagttcttgaatgtcagcattatggcaattccttgcctgtgttcagtggcacctagaggcttggcacctaataccagattaaataatggaagtatggctcttataattgcccgaaacacttctcggccagaatttataaaacacctgaaaagatatgccagtgtaaaaaatcagttcaattttccatttgttgagacttacactgttgaggaagtaaaagttcatccaaggaataatactggtggatataatccagaggaggaggaggatgaaactgcttcagaaaattgtttcccttggaatgtagatggtgatttaatggaagttgcatcagaggtccatattagattgcatccaagacttatcagtctttatggaggaagcatggaagaaatgattccaaagtaa

SEQ ID NO: 9 - DNA, 1506 bp, Homo sapiens
atgccctggaggaggcgcaggaaccgggtgagtgccctggagggcggccgggaggaagaggcgcccccggaggctgccgctgtgcctccggcgctgttaacgtccccgcagcagacggaggcggcggccgagcggattctgctccggggcatcttcgagatcgggagggacagttgtgacgtggtgctgagcgagcgagcactgcggtggcggcccattcagcccgagcgcccggcgggtgattctaagtatgacttgctatgtaaagaagaatttattgaactcaaagacatattctctgtgaaactgaaacggcgttgttctgttaaacagcagagaagtggtactttattaggtatcacactcttcatctgcttgaaaaaggaacaaaataaactaaagaattctacacttgatcttattaatttaagtgaagaccactgtgacatatggtttagacagttcaagaaaatattggcaggctttccaaacagaccgaagtcattaaaaatactccttaacccccaaagtcacaaaaaagaagctacccaggtttattatgagaaggttgaacctctgttgaagcttgcaggaataaaaactgatgtaacaataatggaatatgaagggcacgctctgtcactgcttaaggaatgtgaactccagggatttgatggtgttgtctgtgttggtggagatggatctgctagcgaagtagcccatgctttgcttctgagagctcagaagaatgctgggatggaaacagaccgaatcctgactcctgtcagagcacagcttccacttggcttaataccagcaggatctaccaatgtattggcacattctcttcatggagttcctcatgtgataactgcaacattgcacattataatggggcatgtacagctggtcgacgtctgcaccttcagcaccgctggcaagcttcttcgctttgggttctcagccatgtttggctttggtggaagaactttggctctggcagaaaaatatcgatggatgtcccctaaccaacggagagattttgctgttgttaaggcactggcaaaacttaaggcagaagactgtgaaatatcatttttaccatttaacagctctgatgatgtgcaagaaaggagggcacagggatctcccaaatctgaaatatttaatttcagattaaataatggaagtatggctcttataattgcccgaaacacttctcggccagaatttataaaacacctgaaaagatatgccagtgtaaaaaatcagttcaattttccatttgttgagacttacactgttgaggaagtaaaagttcatccaaggaataatactggtggatataatccagaggaggaggaggatgaaactgcttcagaaaattgtttcccttggaatgtagatggtgacttaatggaagttgcatcagaggtccatattagattgcatccaagacttatcagtctttatggaggaagcatggaagaaatgattccaaagtaa

SEQ ID NO: 10 - protein, 532 aa, Homo sapiens
MPWRRRRNRVSALEGGREEEAPPEAAAVPPALLTSPQQTEAAAERILLRGIFEIGRDSCDVVLSERALRWRPIQPERPAGDSKYDLLCKEEFIELKDIFSVKLKRRCSVKQQRSGTLLGITLFICLKKEQNKLKNSTLDLINLSEDHCDIWFRQFKKILAGFPNRPKSLKILLNPQSHKKEATQVYYEKVEPLLKLAGIKTDVTIMEYEGHALSLLKECELQGFDGVVCVGGDGSASEVAHALLLRAQKNAGMETDRILTPVRAQLPLGLIPAGSTNVLAHSLHGVPHVITATLHIIMGHVQLVDVCTFSTAGKLLRFGFSAMFGFGGRTLALAEKYRWMSPNQRRDFAVVKALAKLKAEDCEISFLPFNSSDDVQERRAQGSPKSDCNDQWQMIQGQFLNVSIMAIPCLCSVAPRGLAPNTRLNNGSMALIIARNTSRPEFIKHLKRYASVKNQFNFPFVETYTVEEVKVHPRNNTGGYNPEEEEDETASENCFPWNVDGDLMEVASEVHIRLHPRLISLYGGSMEEMIPK

SEQ ID NO: 11 - protein, 240 aa, Homo sapiens
MEYEGHALSLLKECELQGFDGVVCVGGDGSASEVAHALLLRAQKNAGMETDRILTPVRAQLPLGLIPAGSTNVLAHSLHGVPHVITATLHIIMGHVQLVDVCTFSTAGKLLRFGFSAMFGFGGRTLALAEKYRWMSPNQRRDFAVVKALAKLKAEDCEISFLPFNSSDDVQERRAQGSPKSDCNDQWQMIQGQFLNVSIMAIPCLCSVAPRGLAPNTRLNNGSMALIIARNTSRPEFIKH

SEQ ID NO: 12 - protein, 379 aa, Homo sapiens
MPWRRRRNRVSALEGGREEEAPPEAAAVPPALLTSPQQTEAAAERILLRGIFEIGRDSCDVVLSERALRWRPIQPERPAGDSKYDLLCKEEFIELKDIFSVKLKRRCSVKQQRSGTLLGITLFICLKKEQNKLKNSTLDLINLSEDHCDIWFRQFKKILAGFPNRPKSLKILLNPQSHKKEATQVYYEKVEPLLKLAGIKTDVTIMEYEGHALSLLKECELQGFDGVVCVGGDGSASEVAHALLLRAQKNAGMETDRILTPVRAQLPLGLIPAGSTNVLAHSLHGVPHVITATLHIIMGHVQLVDVCTFSTAGKLLRFGFSAMFGFGGRTLALAEKYRWMSPNQRRDFAVVKALAKLKAEDCEISFLLPFNSSDDVQER

SEQ ID NO: 13 - protein, 216 aa, Homo sapiens
MPWRRRRNRVSALEGGREEEAPPEAAAVPPALLTSPQQTEAAAERILLRGIFEIGRDSCDVVLSERALRWRPIQPERPAGDSKYDLLCKEEFIELKDIFSVKLKRRCSVKQQRSGTLLGITLFICLKKEQNKLKNSTLDLINLSEDHCDIWFRQFKKILAGFPNRPKSLKILLNPQSHKKEATQVYYEKVEPLLKLAGIKTDVTMSVLVEMDLLAK

SEQ ID NO: 14 - protein, 144 aa, Homo sapiens
MPWRRRRNRVSALEGGREEEAPPEAAAVPPALLTSPQQTEAAAERILLRGIFEIGRDSCDVVLSERALRWRPIQPERPAGDSKYDLLCKEEFIELKDIFSVKLKRRCSVKQQRSGTLLGITLFICLKKDQNKLKILHLILLLIK

SEQ ID NO: 15 - protein, 501 aa, Homo sapiens
MPWRRRRNRVSALEGGREEEAPPEAAAVPPALLTSPQQTEAAAERILLRGIFEIGRDSCDVVLSERALRWRPIQPERPAGDSKYDLLCKEEFIELKDIFSVKLKRRCSVKQQRSGTLLGITLFICLKKEQNKLKNSTLDLINLSEDHCDIWFRQFKKILAGFPNRPKSLKILLNPQSHKKEATQVYYEKVEPLLKLAGIKTDVTIMEYEGHALSLLKECELQGFDGVVCVGGDGSASEVAHALLLRAQKNAGMETDRILTPVRAQLPLGLIPAGSTNVLAHSLHGVPHVITATLHIIMGHVQLVDVCTFSTAGKLLRFGFSAMFGFGGRTLALAEKYRWMSPNQRRDFAVVKALAKLKAEDCEISFLPFNSSDDVQERRAQGSPKSEIFNFRLNNGSMALIIARNTSRPEFIKHLKRYASVKNQFNFPFVETYTVEEVKVHPRNNTGGYNPEEEEDETASENCFPWNVDGDLMEVASEVHIRLHPRLISLYGGSMEEMIPK